(19) United States
(12) Patent Application Publication
(10) Pub. No.: US 2021/0292734 A1
(43) Pub. Date: Sep. 23, 2021

(54) INCREASED ALCOHOL PRODUCTION FROM YEAST PRODUCING AN INCREASED AMOUNT OF ACTIVE CRZ1 PROTEIN

(71) Applicant: DANISCO US INC, Palo Alto, CA (US)

(72) Inventors: Joseph Frederich Tuminello, WILMINGTON, DE (US); QUINN QUN ZHU, WEST CHESTER, PA (US)

(21) Appl. No.: 17/263,679

(22) PCT Filed: Jul. 26, 2019
(86) PCT No.: PCT/US19/43694

§ 371 Date: Jan. 27, 2021

Related U.S. Application Data
(60) Provisional application No. 62/703,952, filed on Jul. 27, 2018.

(51) Int. Cl. C12N 9/88; C07K 14/39; C07K 14/395; C12N 9/10; C12N 9/02; C12P 7/06

(52) U.S. Cl. CPC C12N 9/88; C07K 14/39; C07K 14/395; C12Y 401/02009; C12P 7/06; C12Y 203/01008; C12N 9/0008; C12Y 102/0101; C12N 9/1029

(57) ABSTRACT

Described are compositions and methods relate to modified yeast that, in addition to native endogenous CRZ1, produces a modified CRZ1 transcriptional activator involved in the calcineurin stress response pathway. Such yeast is well suited for use in fuel alcohol production to increase yield.

### TECHNICAL FIELD

The present compositions and methods relate to modified yeast that produces an increased amount of an active, variant-CRZ1 transcriptional activators involved in the calcineurin stress response pathway. Such yeast is well suited for use in fuel alcohol production to increase yield.

### BACKGROUND

Many countries make fuel alcohol from fermentable substrates, such as corn starch, sugar cane, cassava, and molasses. According to the Renewable Fuel Association (Washington D.C., United States), 2015 fuel ethanol production was close to 15 billion gallons in the United States, alone.

Butanol is an important industrial chemical and drop-in fuel component with a variety of applications including use as a renewable fuel additive, a feedstock chemical in the plastics industry, and a food-grade extractant in the food and flavor industry. Accordingly, there is a high demand for alcohols such as butanol and isobutanol, as well as for efficient and environmentally-friendly production methods.

In view of the large amount of alcohol produced in the world, even a minor increase in the efficiency of a fermenting organism can result in a tremendous increase in the amount of available alcohol. Accordingly, the need exists for organisms that are more efficient at producing alcohol.

### SUMMARY

Described are compositions and methods relating to modified yeast that produces a variant CRZ1 transcriptional activator with respect to otherwise-identical parental yeast. Aspects and embodiments of the compositions and methods are described in the following, independently-numbered, paragraphs.

1. In one aspect, modified yeast cells derived from parental yeast cells are provided, the modified cells comprising a genetic alteration that causes the cells to produce an increased amount of active CRZ1 polypeptides compared to the parental cells, wherein the modified cells produce during fermentation an increased amount of alcohol compared to the amount of alcohol produced by the parental cells under identical fermentation conditions.

2. In some embodiments of the modified cells of paragraph 1, the active CRZ1 polypeptides exhibit reduced phosphorylation compared to native CRZ1 polypeptides under identical fermentation conditions.

3. In some embodiments of the modified cells of paragraph 1 or 2, the active CRZ1 polypeptides include a reduced number of amino acid residues capable of phosphorylation compared to the amino acid residues in native CRZ1 polypeptides.

4. In some embodiments of the modified cells of paragraph 3, the active CRZ1 polypeptides include a reduced number of serine residues capable of phosphorylation compared to the amino acid residues in native CRZ1 polypeptides.

5. In some embodiments of the modified cells of any of paragraphs 1-4, the amount of increase in the expression of the modified CRZ1 mutant polypeptides is at least about 500%, at least 1,000%, at least 1,500%, or even at least 2,000%, compared to the level expression of native CRZ1 polypeptides in the parental cells grown under equivalent conditions.

6. In some embodiments of the modified cells of any of paragraphs 1-5, the cells further comprising one or more genes of the phosphoketolase pathway.

7. In some embodiments of the modified cells of paragraph 6, the genes of the phosphoketolase pathway are selected from the group consisting of phosphoketolase, phosphotransacetylase and acetylating acetyl dehydrogenase.

8. In some embodiments of the modified cells of any of paragraphs 1-7, the cells further comprise an exogenous gene encoding a carbohydrate processing enzyme.

9. In some embodiments, the modified cells of any of paragraphs 1-8, further comprise an alteration in the glycerol pathways and/or the acetyl-CoA pathway.

10. In some embodiments, the modified cells of any of paragraphs 1-9, further comprise an alternative pathway for making alcohol.

11. In some embodiments of the modified cells of any of paragraphs 1-10, the cells are of a Saccharomyces spp.

12. In some embodiments of the modified cells of any of paragraphs 1-11, the alcohol is ethanol.

13. In another aspect, a method for increasing the production of alcohol from yeast cells grown on a carbohydrate substrate is provided, comprising: introducing into parental yeast cells a genetic alteration that increases the production of active CRZ1 polypeptides compared to the amount produced in the parental cells.

14. In some embodiments of the method of paragraph 13, the active CRZ1 polypeptides exhibit reduced phosphorylation compared to native CRZ1 polypeptides under identical fermentation conditions.

15. In some embodiments of the method of paragraph 13 or 14, the active CRZ1 polypeptides include a reduced number of amino acid residues capable of phosphorylation compared to the amino acid residues in native CRZ1 polypeptides.

16. In some embodiments of the method of paragraph 15, the active CRZ1 polypeptides include a reduced number of serine residues capable of phosphorylation compared to the amino acid residues in native CRZ1 polypeptides.

17. In some embodiments of the method of any of paragraphs 13-16, the genetic alteration results in the variant yeast of any of paragraphs 1-11, or variants, thereof.

These and other aspects and embodiments of the present compositions and methods will be apparent from the description, including the accompanying Drawings.

### BRIEF DESCRIPTION OF THE DRAWING

FIG. 1 is a Clustal W alignment comparing the native CRZ1 gene (SEQ ID NO: 3; bottom) to the genetically modified CRZ1 variant gene (SEQ ID NO: 4; top) containing a silent mutation at nucleotide position 39 resulting in the deletion of a SpeI restriction site and further containing nucleotide changes resulting in alanine residues at positions 68, 69, 77 and 78 replacing serine residues. Nucleotide modifications are shown in the Clustal W alignment of FIG. 1. Differences in the sequences are shown in bold typeface.

FIG. 2 is a Clustal W alignment comparing the native CRZ1 polypeptide (SEQ ID NO: 1) to the genetically modified CRZ1 variant polypeptide (SEQ ID NO: 2). The serine to alanine substitutions at positions 68, 69, 77 and 78 are shown in bold typeface.

### DETAILED DESCRIPTION

### I. Overview

The present compositions and methods relate to modified yeast that produces a variant CRZ1 transcriptional activator involved in the calcineurin stress response pathway. The native CRZ1 gene in Saccharomyces cerevisiae is constitutively transcribed, translated and folded into a protein (Crz1p), a member of the zinc-finger family of transcription factors. Crz1p contains a serine-rich region (SRR) that, under non-stress conditions, is phosphorylated. Under non-stressful environmental conditions, phosphorylated Crz1p is localized to the cytoplasm. When cells are subjected to environmental stresses, such as a lack of nutrients, thermal, chemical or osmotic stress, and the like, Crz1p is dephosphorylated by the induced calcineurin complex and the calcineurin stress response pathway is activated. Dephosphorylated Crz1p is translocated into the nucleus where it affects genes that act in cellular stress response. In this manner, dephosphorylated Crz1p is considered the active form of the molecule.

While in the nucleus the state of Crz1p phosphorylation changes rapidly with kinases rephosphorylating the serine residues in the SRR causing rapid export of Crz1p from the nucleus. Kinetically, export is favored over import and the existence of Crz1p in the dephosphorylated state is fleeting.

While the presence of alcohol is known to induce the calcineurin stress response pathway, it was heretofore unknown that the expression of a modified CRZ1 protein in addition to native endogenous CRZ1 would result in increased alcohol tolerance. This observation suggests that the calcineurin stress response pathway in yeast is not naturally optimized for the stress of alcohol production, even in commercial yeast selected specifically for this purpose. Various aspects and embodiments of present composition and methods are described in detail, herein.

### II. Definitions

Prior to describing the modified cells and methods of use in detail, the following terms are defined for clarity. Terms not defined should be accorded their ordinary meanings as used in the relevant art.

As used herein, the term “alcohol” refers to an organic compound in which a hydroxyl functional group (—OH) is bound to a saturated carbon atom.

As used herein, “yeast cells,” “yeast strains” or simply “yeast” refer to organisms from the phyla Ascomycota and Basidiomycota. Exemplary yeast is budding yeast from the order Saccharomycetales. Particular examples of yeast are Saccharomyces spp., including but not limited to S. cerevisiae. Yeast include organisms used for the production of fuel alcohol as well as organisms used for the production of potable alcohol, including specialty and proprietary yeast strains used to make distinctive-tasting beers, wines, and other fermented beverages.

As used herein, the phrase “variant yeast cells,” “modified yeast cells,” or similar phrases (see above), refer to yeast that include genetic modifications and characteristics described herein. Variant/modified yeast does not include naturally occurring yeast.

As used herein, the terms “polypeptide” and “protein” (and their respective plural forms) are used interchangeably to refer to polymers of any length comprising amino acid residues linked by peptide bonds. The conventional one-letter or three-letter codes for amino acid residues are used herein and all sequence are presented from an N-terminal to C-terminal direction. The polymer can be linear or branched, it can comprise modified amino acids, and can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, functionally and/or structurally similar proteins are considered to be “related proteins.” Such proteins can be derived from organisms of different genera and/or species, or even different classes of organisms (e.g., bacteria and fungi). Related proteins also encompass homologs determined by primary sequence analysis, determined by secondary or tertiary structure analysis, or determined by immunological cross-reactivity or function.

As used herein, the term “homologous protein” or “homolog” refers to a protein that has similar activity and/or structure to a reference protein. It is not intended that homologs necessarily be evolutionarily related. Thus, it is intended that the term encompass the same, similar, or corresponding enzyme(s) (i.e., in terms of structure and function) obtained from different organisms. In some embodiments, it is desirable to identify a homolog that has a quaternary, tertiary and/or primary structure similar to the reference protein. In some embodiments, homologous proteins induce similar immunological response(s) as a reference protein. In some embodiments, homologous proteins are engineered to produce enzymes with desired activity(ies).

The degree of homology between sequences can be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, Wis.); and Devereux et al. (1984) Nucleic Acids Res. 12:387-95).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-60). The method is similar to that described by Higgins and Sharp ((1989) CABIOS 5:151-53). Useful PILEUP parameters including a default gap weight of 3.00, a default gap length weight of 0.10, and weighted end gaps. Another example of a useful algorithm is the BLAST algorithm, described by Altschul et al. ((1990) J. Mol. Biol. 215:403-10) and Karlin et al. ((1993) Proc. Natl. Acad. Sci. USA 90:5873-87). One particularly useful BLAST program is the WU-BLAST-2 program (see, e.g., Altschul et al. (1996) Meth. Enzymol. 266:460-80). Parameters “W,” “T,” and “X” determine the sensitivity and speed of the alignment. The BLAST program uses as defaults a word-length (W) of 11, the BLOSUM62 scoring matrix (see, e.g., Henikoff and Henikoff (1989) Proc. Natl. Acad. Sci. USA 89:10915) alignments (B) of 50, expectation (E) of 10, M′S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Percent sequence identity is calculated using CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF |

Another indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, the term “gene” is synonymous with the term “allele” in referring to a nucleic acid that encodes and directs the expression of a protein or RNA. Vegetative forms of filamentous fungi are generally haploid, therefore a single copy of a specified gene (i.e., a single allele) is sufficient to confer a specified phenotype.

As used herein, “expressing a polypeptide” and similar terms, refer to the cellular process of producing a polypeptide using the translation machinery (e.g., ribosomes) of the cell.

As used herein, “overexpressing a polypeptide,” “increasing the expression of a polypeptide,” and similar terms, refer to expressing a polypeptide at higher-than-normal levels compared to those observed with parental or “wild-type cells that do not include a specified genetic modification.

As used herein, an “expression cassette” refers to a DNA fragment that includes promoter, an amino acid coding sequence, terminator, and other nucleic acid sequence needed to allow the encoded polypeptide to be produced in a cell. Expression cassettes can be exogenous (i.e., introduced into a cell) or endogenous (i.e., extant in a cell).

As used herein, the terms “wild-type” and “native” are used interchangeably and refer to genes proteins or strains found in nature.

As used herein, the term “protein of interest” refers to a polypeptide that is desired to be expressed in modified yeast. Such a protein can be an enzyme, a factor, a co-factor, a substrate-binding protein, a surface-active protein, a structural protein, a selectable marker, or the like, and can be expressed at high levels. The protein of interest is encoded by a modified endogenous gene or a heterologous gene (i.e., gene of interest”) relative to the parental strain. The protein of interest can be expressed intracellularly or as a secreted protein.

As used herein, the term “native” is synonymous with wild-type and non-genetically modified.

As used herein, the terms “genetic manipulation” and “genetic alteration” are used interchangeably and refer to the alteration/change of a nucleic acid sequence. The alteration can include but is not limited to a substitution, deletion, insertion or chemical modification of at least one nucleic acid in the nucleic acid sequence.

As used herein, an “active polypeptide/protein” possesses a defined activity.

As used herein, “genetically-modified,” particularly with respect to a CRZ1 gene, transcribed mRNA or active modified CRZ1 protein, refers to a version of the gene, mRNA or protein that has been genetically manipulated by human intervention.

As used herein, “aerobic fermentation” refers to growth in the presence of oxygen.

As used herein, “anaerobic fermentation” refers to growth in the absence of oxygen.

As used herein, the singular articles “a,” “an,” and “the” encompass the plural referents unless the context clearly dictates otherwise. All references cited herein are hereby incorporated by reference in their entirety. The following abbreviations/acronyms have the following meanings unless otherwise specified:

| | |
|---|---|
| ° C. | degrees Centigrade |
| AA | α-amylase |
| bp | base pairs |
| DNA | deoxyribonucleic acid |
| DP | degree of polymerization |
| ds or DS | dry solids |
| EtOH | ethanol |
| g or gm | gram |
| g/L | grams per liter |
| GA | glucoamylase |
| GAU/g ds | glucoamylase units per gram dry solids |
| H₂O | water |
| HPLC | high performance liquid chromatography |
| hr or h | hour |
| kg | kilogram |
| M | molar |
| mg | milligram |
| mL or ml | milliliter |
| ml/min | milliliter per minute |
| mM | millimolar |
| N | normal |
| nm | nanometer |
| PCR | polymerase chain reaction |
| ppm | parts per million |
| RNA | ribonucleic acid |
| Δ | relating to a deletion |
| μg | microgram |
| μL and μl | microliter |
| μM | micromolar |

III. Modified Yeast Cells having Modified CRZ1 Expression

In one aspect, modified yeast cells are provided, the modified cells having a genetic alteration that results in the production of modified CRZ1 polypeptides that are in addition to native endogenous CRZ1 polypeptides compared to otherwise-identical parental cells. The amino acid sequence of the exemplified active S. cerevisiae S288c CRZ1 polypeptide (i.e., EMBL Accession No. NP_014371) is shown, below, as SEQ ID NO: 1. Wild-type serine residues mentioned in the accompanying text are underlined:

MSFSNGNMASYMTSSNGEEQSINNKNDIDDNSAYRRNNFRNSSNSGSHTF
QLSDLDLDVDMRMDAANSSEKISKNLSSGIPDSFDSNVNSLLSPSSGSYS
ADLNYQSLYKPDLPQQQLQQQQLQQQQQQQQQQQQQQQKQTPTLKVEQSD
TFQWDDILTPADNQHRPSLTNQFLSPRSNYDGTTRSSGIDSNYSDTESNY
HTPYLYPQDLVSSPAMSHLTANNDDFDDLLSVASMNSNYLLPVNSHGYKH
ISNLDELDDLLSLTYSDNNLLSASNNSDFNNSNNGIINTADTQNSTIAIN
KSKVGTNQKMLLTIPTSSTPSPSTHAAPVTPIISIQEFNEGHFPVKNEDD
GTLQLKVRDNESYSATNNNNLLRPDDNDYNNEALSDIDRSFEDIINGRKL
KLKKSRRRSSQTSNNSFTSRRSSRSRSISPDEKAKSISANREKLLEMADL
LPSSENDNNRERYDNDSKTSYNTINSSNFNEDNNNNNLLTSKPKIESGIV
NIKNELDDTSKDLGILLDIDSLGQFEQKVGFKNDDNHENNDNGTFSVKKN
DNLEKLDSVTNNRKNPANFACDVCGKKFTRPYNLKSHLRTHTNERPFICS
ICGKAFARQHDRKRHEDLHTGKKRYVCGGKLKDGKPWGCGKKFARSDALG
RHFKTESGRRCITPLYEEARQEKSGQES

The amino acid sequence of the exemplified genetically modified S. cerevisiae S288c CRZ1 polypeptide is shown, below, as SEQ ID NO: 2. Alanine substitutions mentioned in the accompanying text are underlined:

MSFSNGNMASYMTSSNGEEQSINNKNDIDDNSAYRRNNFRNSSNSGSHTF
QLSDLDLDVDMRMDAANAAEKISKNLAAGIPDSFDSNVNSLLSPSSGSYS
ADLNYQSLYKPDLPQQQLQQQQLQQQQQQQQQQQQQQQKQTPTLKVEQSD
TFQWDDILTPADNQHRPSLTNQFLSPRSNYDGTTRSSGIDSNYSDTESNY
HTPYLYPQDLVSSPAMSHLTANNDDFDDLLSVASMNSNYLLPVNSHGYKH
ISNLDELDDLLSLTYSDNNLLSASNNSDFNNSNNGIINTADTQNSTIAIN
KSKVGTNQKMLLTIPTSSTPSPSTHAAPVTPIISIQEFNEGHFPVKNEDD
GTLQLKVRDNESYSATNNNNLLRPDDNDYNNEALSDIDRSFEDIINGRKL
KLKKSRRRSSQTSNNSFTSRRSSRSRSISPDEKAKSISANREKLLEMADL
LPSSENDNNRERYDNDSKTSYNTINSSNFNEDNNNNNLLTSKPKIESGIV
NIKNELDDTSKDLGILLDIDSLGQFEQKVGFKNDDNHENNDNGTFSVKKN
DNLEKLDSVTNNRKNPANFACDVCGKKFTRPYNLKSHLRTHTNERPFICS
ICGKAFARQHDRKRHEDLHTGKKRYVCGGKLKDGKPWGCGKKFARSDALG
RHFKTESGRRCITPLYEEARQEKSGQES

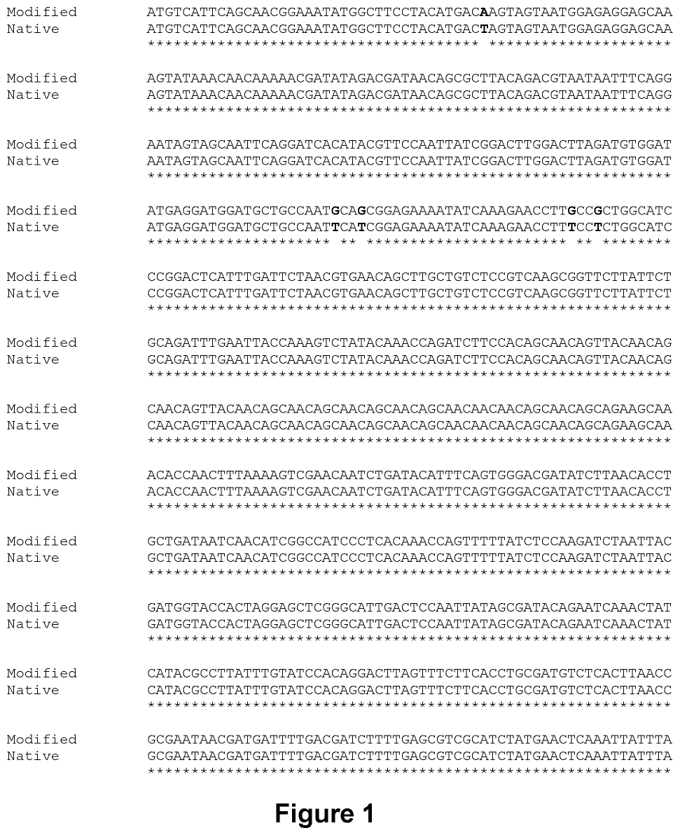

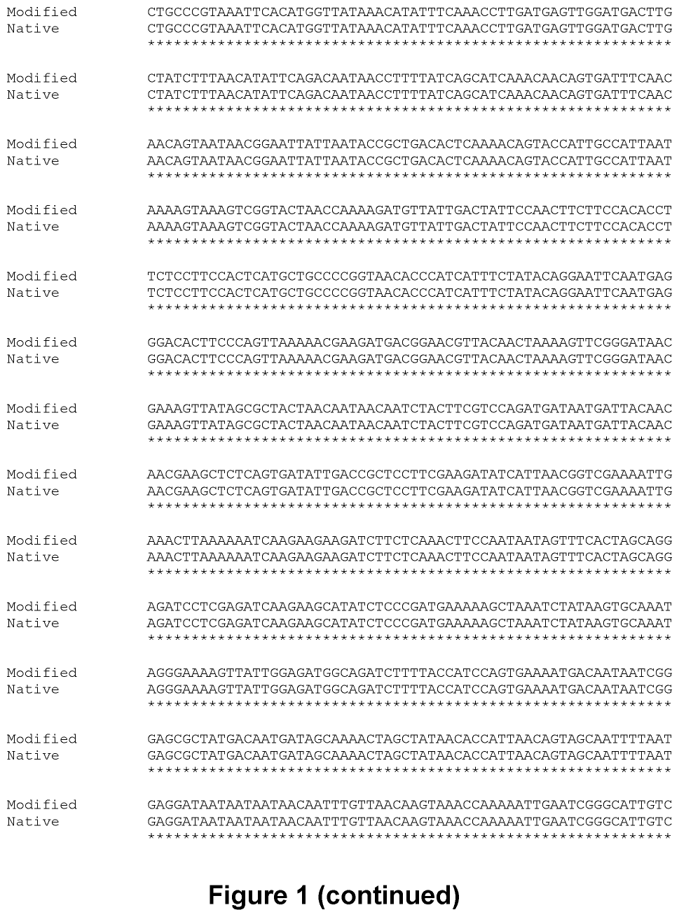

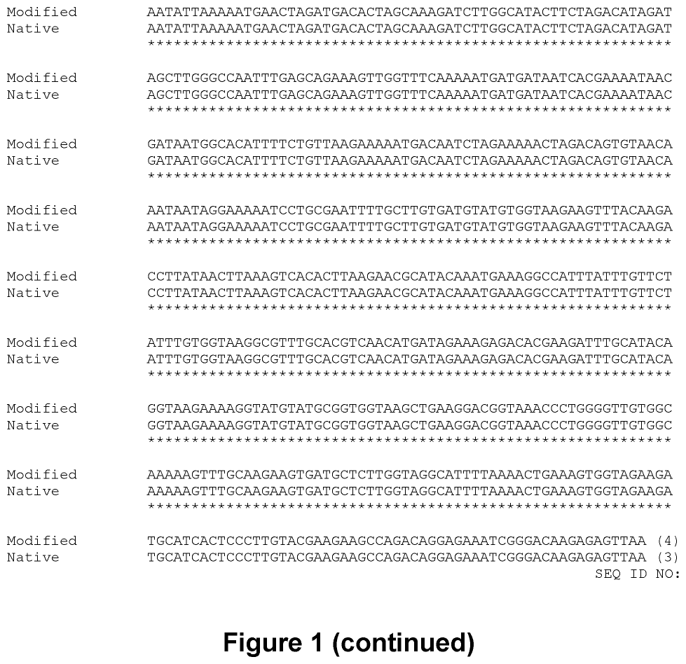

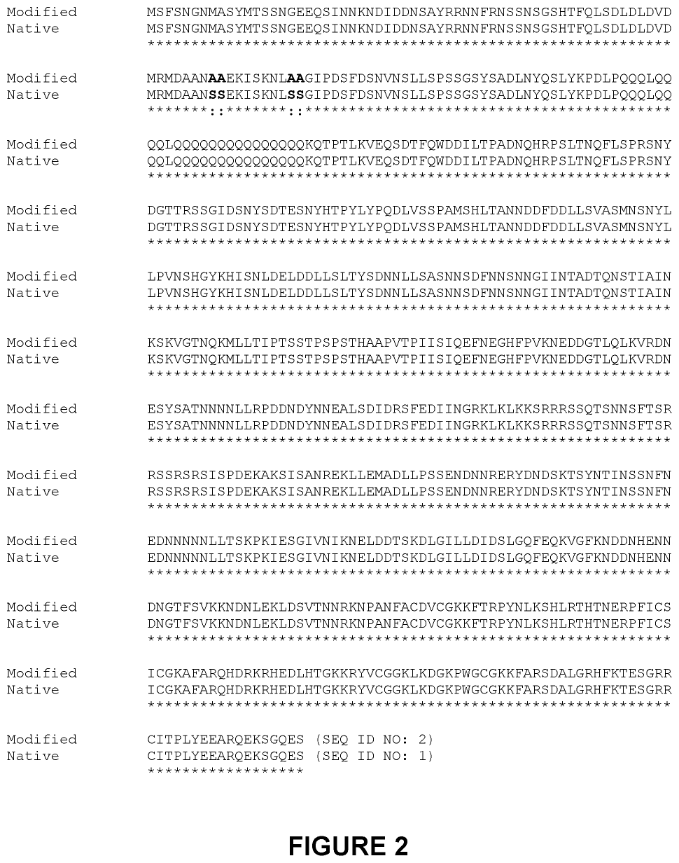

The genetically modified gene includes a silent mutation at nucleotide position 39 resulting in the deletion of a SpeI restriction site and further includes nucleotide changes resulting in serine residues at positions 68, 69, 77 and 78 being replace alanine residues. Nucleotide modifications are shown in the Clustal W alignment of FIG. 1. The differences between the native and genetically-modified polypeptides are shown in the Clustal W alignment of FIG. 2. Without being bound to a theory, it is believed that the substitution of serine residues to alanine removes phosphorylation sites, thereby mimicking dephophorylation of the native CRZ1 polypeptide, resulting in nuclear localization and enhanced stress response. Other residue capable of being phosphorylated, particularly those in the SRR, can presumably also be substituted instead of or in addition to those exemplified, with similar effect.

In some embodiments, genetically-modified CRZ1 polypeptides are expressed in combination with, and in addition to, native CRZ1 polypeptides. In some embodiments, the genetically-modified CRZ1 polypeptides are expressed in place of, i.e. in the absence of, native CRZ1 polypeptides. In some embodiments, the modified CRZ1 polypeptide is expressed in excess over the native polypeptides. In some embodiments, the increase in the expression of the CRZ1 mutant polypeptides is at least at least 500%, at least 1,000%, at least 1,500%, or even at least 2,000%, or more, compared to the level expression of the native polypeptides in the parental cells grown under equivalent conditions.

In some embodiments, the increase in alcohol production by the modified cells is an increase of at least 0.3%, at least, 0.5%, at least 0.6%, at least 0.7%, at least 0.8%, at least 0.9%, at least 1.0%, at least 1.1%, at least 1.2%, at least 1.3%, at least 1.4%, at least 1.5%, at least 1.6%, at least 1.7%, at least 1.8%, at least 1.9%, at least 2.0% or more, compared to the amount of alcohol produced by parental cells grown under the same conditions.

Preferably, modified CRZ1 production is achieved by genetic manipulation using sequence-specific molecular biology techniques, as opposed to chemical mutagenesis, which is generally not targeted to specific nucleic acid sequences. However, chemical mutagenesis is not excluded as a method for making modified yeast cells. Modified CRZ1 production can also be achieved by classic evolution using designed screening techniques.

In some embodiments, the parental cell that is already modified to include a gene of interest, such as a gene encoding a selectable marker, carbohydrate-processing enzyme, or other polypeptide. In some embodiments, a gene of introduced is subsequently introduced into the modified cells.

IV. Combination of Modified CRZ1 Production with Genes of an Exogenous PKL Pathway

Expression of modified CRZ1 can be combined with expression of genes in the PKL pathway to increase the production of ethanol.

Engineered yeast cells having a heterologous PKL pathway have been previously described in WO2015148272 (Miasnikov et al.). These cells express heterologous phosphoketolase (PKL), phosphotransacetylase (PTA) and acetylating acetyl dehydrogenase (AADH), optionally with other enzymes, to channel carbon flux away from the glycerol pathway and toward the synthesis of acetyl-CoA, which is then converted to ethanol. Such modified cells are capable of increased ethanol production in a fermentation process when compared to otherwise-identical parent yeast cells.

V. Combination of Modified CRZ1 Production with Other Mutations that Affect Alcohol Production and/or Glycerol Reduction

In some embodiments, in addition to expressing modified CRZ1 polypeptides, the present modified yeast cells include additional modifications that affect ethanol production.

The modified cells may further include mutations that result in attenuation of the native glycerol biosynthesis pathway and/or reuse glycerol pathway, which are known to increase alcohol production. Methods for attenuation of the glycerol biosynthesis pathway in yeast are known and include reduction or elimination of endogenous NAD-dependent glycerol 3-phosphate dehydrogenase (GPD) or glycerol phosphate phosphatase activity (GPP), for example by disruption of one or more of the genes GPD1, GPD2, GPP1 and/or GPP2. See, e.g., U.S. Pat. No. 9,175,270 (Elke et al.), U.S. Pat. No. 8,795,998 (Pronk et al.) and U.S. Pat. No. 8,956,851 (Argyros et al.). Methods to enhance the reuse glycerol pathway by over expression of glycerol dehydrogenase (GCY1) and dihydroxyacetone kinase (DAK1) to convert glycerol to dihydroxyacetone phosphate (Zhang et al. (2013) J. Ind. Microbiol. Biotechnol. 40:1153-60).

The modified yeast may further feature increased acetyl-CoA synthase (also referred to acetyl-CoA ligase) activity to scavenge (i.e., capture) acetate produced by chemical or enzymatic hydrolysis of acetyl-phosphate (or present in the culture medium of the yeast for any other reason) and converts it to Ac-CoA. This avoids the undesirable effect of acetate on the growth of yeast cells and may further contribute to an improvement in alcohol yield. Increasing acetyl-CoA synthase activity may be accomplished by introducing a heterologous acetyl-CoA synthase gene into cells, increasing the expression of an endogenous acetyl-CoA synthase gene and the like. A particularly useful acetyl-CoA synthase for introduction into cells can be obtained from Methanosaeta concilii (UniProt/TrEMBL Accession No.: WP_013718460). Homologs of this enzymes, including enzymes having at least 85%, at least 90%, at least 92%, at least 95%, at least 97%, at least 98% and even at least 99% amino acid sequence identity to the aforementioned acetyl-CoA synthase from Methanosaeta concilii, are also useful in the present compositions and methods.

In some embodiments, the modified cells may further include a heterologous gene encoding a protein with NAD⁺-dependent acetylating acetaldehyde dehydrogenase activity and/or a heterologous gene encoding a pyruvate-formate lyase. The introduction of such genes in combination with attenuation of the glycerol pathway is described, e.g., in U.S. Pat. No. 8,795,998 (Pronk et al.). In some embodiments of the present compositions and methods the yeast expressly lacks a heterologous gene(s) encoding an acetylating acetaldehyde dehydrogenase, a pyruvate-formate lyase or both.

In some embodiments, the present modified yeast cells may further overexpress a sugar transporter-like (STL1) polypeptide (see, e.g., Ferreira et al. (2005) Mol Biol Cell 16:2068-76; Dušková et al. (2015) Mol Microbiol 97:541-59 and WO 2015023989 A1) to increase ethanol production and reduce acetate.

In some embodiments, the present modified yeast cells may further overexpress a polyadenylate-binding protein, e.g., PAB1, to increase alcohol production and reduce acetate production.

In some embodiments, the present modified yeast cells further include a butanol biosynthetic pathway. In some embodiments, the butanol biosynthetic pathway is an isobutanol biosynthetic pathway. In some embodiments, the isobutanol biosynthetic pathway comprises a polynucleotide encoding a polypeptide that catalyzes a substrate to product conversion selected from the group consisting of: (a) pyruvate to acetolactate; (b) acetolactate to 2,3-dihydroxyisovalerate; (c) 2,3-dihydroxyisovalerate to 2-ketoisovalerate; (d) 2-ketoisovalerate to isobutyraldehyde; and (e) isobutyraldehyde to isobutanol. In some embodiments, the isobutanol biosynthetic pathway comprises polynucleotides encoding polypeptides having acetolactate synthase, keto acid reductoisomerase, dihydroxy acid dehydratase, ketoisovalerate decarboxylase, and alcohol dehydrogenase activity.

In some embodiments, the modified yeast cells comprising a butanol biosynthetic pathway further comprise a modification in a polynucleotide encoding a polypeptide having pyruvate decarboxylase activity. In some embodiments, the yeast cells comprise a deletion, mutation, and/or substitution in an endogenous polynucleotide encoding a polypeptide having pyruvate decarboxylase activity. In some embodiments, the polypeptide having pyruvate decarboxylase activity is selected from the group consisting of: PDC1, PDC5, PDC6, and combinations thereof. In some embodiments, the yeast cells further comprise a deletion, mutation, and/or substitution in one or more endogenous polynucleotides encoding FRA2, ALD6, ADH1, GPD2, BDH1, and YMR226C.

VI. Combination of Modified CRZ1 Production with Other Beneficial Mutations

In some embodiments, in addition to expression of modified CRZ1 polypeptides, optionally in combination with other genetic modifications that benefit alcohol production, the present modified yeast cells further include any number of additional genes of interest encoding proteins of interest. Additional genes of interest may be introduced before, during, or after genetic manipulations that result in the increased production of modified CRZ1 polypeptides.

Proteins of interest, include selectable markers, carbohydrate-processing enzymes, and other commercially-relevant polypeptides, including but not limited to an enzyme selected from the group consisting of a dehydrogenase, a transketolase, a phosphoketolase, a transaldolase, an epimerase, a phytase, a xylanase, a β-glucanase, a phosphatase, a protease, an α-amylase, a β-amylase, a glucoamylase, a pullulanase, an isoamylase, a cellulase, a trehalase, a lipase, a pectinase, a polyesterase, a cutinase, an oxidase, a transferase, a reductase, a hemicellulase, a mannanase, an esterase, an isomerase, a pectinases, a lactase, a peroxidase and a laccase. Proteins of interest may be secreted, glycosylated, and otherwise-modified.

### VII. Use of the Modified Yeast for Increased Alcohol Production

The present compositions and methods include methods for increasing alcohol production and/or reducing glycerol production, in fermentation reactions. Such methods are not limited to a particular fermentation process. The present engineered yeast is expected to be a “drop-in” replacement for convention yeast in any alcohol fermentation facility. While primarily intended for fuel alcohol production, the present yeast can also be used for the production of potable alcohol, including wine and beer.

### VIII. Yeast Cells Suitable for Modification

Yeasts are unicellular eukaryotic microorganisms classified as members of the fungus kingdom and include organisms from the phyla Ascomycota and Basidiomycota. Yeast that can be used for alcohol production include, but are not limited to, Saccharomyces spp., including S. cerevisiae, as well as Kluyveromyces, Lachancea and Schizosaccharomyces spp. Numerous yeast strains are commercially available, many of which have been selected or genetically engineered for desired characteristics, such as high alcohol production, rapid growth rate, and the like. Some yeasts have been genetically engineered to produce heterologous enzymes, such as glucoamylase or a-amylase.

### IX. Substrates and Products

Alcohol production from a number of carbohydrate substrates, including but not limited to corn starch, sugar cane, cassava, and molasses, is well known, as are innumerable variations and improvements to enzymatic and chemical conditions and mechanical processes. The present compositions and methods are believed to be fully compatible with such substrates and conditions.

Alcohol fermentation products include organic compound having a hydroxyl functional group (—OH) is bound to a carbon atom. Exemplary alcohols include but are not limited to methanol, ethanol, n-propanol, isopropanol, n-butanol, isobutanol, n-pentanol, 2-pentanol, isopentanol, and higher alcohols. The most commonly made fuel alcohols are ethanol, and butanol.

These and other aspects and embodiments of the present yeast strains and methods will be apparent to the skilled person in view of the present description. The following examples are intended to further illustrate, but not limit, the compositions and methods.

### EXAMPLES

### Example 1

### Materials and Methods

### Liquefact Preparation

Liquefact (ground corn slurry) was prepared by adding 600 ppm of urea, 0.124 SAPU/g ds FERMGEN™ 2.5× (acid fungal protease), 0.33 GAU/g ds variant Trichoderma glucoamylase (TrGA) and 1.46 SSCU/g ds Aspergillus α-amylase, adjusted to a pH of 4.8.

### AnKom Assays

300 μL of concentrated yeast overnight culture was added to each of a number ANKOM bottles filled with 30 g prepared liquefact for a final OD of 0.3. The bottles were then incubated at 32° C. with shaking (150 RPM) for 55 hours.

### HPLC Analysis

Samples from serum vial and AnKom experiments were collected in Eppendorf tubes by centrifugation for 12 minutes at 14,000 RPM. The supernatants were filtered with 0.2 μM PTFE filters and then used for HPLC (Agilent Technologies 1200 series) analysis with the following conditions: Bio-Rad Aminex HPX-87H columns, running temperature of 55° C. 0.6 ml/min isocratic flow 0.01 N H₂SO₄, 2.5 μL injection volume. Calibration standards were used for quantification of the of acetate, ethanol, glycerol, and glucose. Samples from shake flasks experiments were collected in Eppendorf tubes by centrifugation for 15 minutes at 14,000 RPM. The supernatants were diluted by a factor of 11 using 5 mM H₂SO₄ and incubated for 5 min at 95° C. Following cooling, samples were filtered with 0.2 μM Corning FiltrEX CLS3505 filters and then used for HPLC analysis. 10 μl was injected into an Agilent 1200 series HPLC equipped with a refractive index detector. The separation column used was a Phenomenex Rezex-RFQ Fast Acid H+ (8%) column. The mobile phase was 5 mM H₂SO₄, and the flow rate was 1.0 mL/min at 85° C. HPLC Calibration Standard Mix from Bion Analytical was used for quantification of the of acetate, ethanol, glycerol, and glucose. Unless otherwise specified, all values are expressed in g/L.

### Example 2

### Generation of a Modified “Active” CRZ1 Gene

The CRZ1 coding sequence from S. cerevisiae S288c was synthesized in a genetically-modified form by changing a thymine nucleotide with an adenine nucleotide at position 39 resulting in a silent mutation that deletes a SpeI restriction site and changing thymine nucleotides for guanine nucleotides at positions 202, 205, 229 and 232 resulting alanine residues replacing serine residues in the SRR. The modified CRZ1 gene was named CRZ1.1.2. The native nucleic acid sequence alignment and polypeptide sequence alignment showing the difference between the two genes are illustrated in FIGS. 1 and 2, respectively. The genetically modified gene is represented by SEQ ID NO: 4, shown, below, with mutations underlined:

ATGTCATTCAGCAACGGAAATATGGCTTCCTACATGACAAGTAGTAATGG
AGAGGAGCAAAGTATAAACAACAAAAACGATATAGACGATAACAGCGCTT
ACAGACGTAATAATTTCAGGAATAGTAGCAATTCAGGATCACATACGTTC
CAATTATCGGACTTGGACTTAGATGTGGATATGAGGATGGATGCTGCCAA
TGCAGCGGAGAAAATATCAAAGAACCTTGCCGCTGGCATCCCGGACTCAT
TTGATTCTAACGTGAACAGCTTGCTGTCTCCGTCAAGCGGTTCTTATTCT
GCAGATTTGAATTACCAAAGTCTATACAAACCAGATCTTCCACAGCAACA
GTTACAACAGCAACAGTTACAACAGCAACAGCAACAGCAACAGCAACAAC
AACAGCAACAGCAGAAGCAAACACCAACTTTAAAAGTCGAACAATCTGAT
ACATTTCAGTGGGACGATATCTTAACACCTGCTGATAATCAACATCGGCC
ATCCCTCACAAACCAGTTTTTATCTCCAAGATCTAATTACGATGGTACCA
CTAGGAGCTCGGGCATTGACTCCAATTATAGCGATACAGAATCAAACTAT
CATACGCCTTATTTGTATCCACAGGACTTAGTTTCTTCACCTGCGATGTC
TCACTTAACCGCGAATAACGATGATTTTGACGATCTTTTGAGCGTCGCAT
CTATGAACTCAAATTATTTACTGCCCGTAAATTCACATGGTTATAAACAT
ATTTCAAACCTTGATGAGTTGGATGACTTGCTATCTTTAACATATTCAGA
CAATAACCTTTTATCAGCATCAAACAACAGTGATTTCAACAACAGTAATA
ACGGAATTATTAATACCGCTGACACTCAAAACAGTACCATTGCCATTAAT
AAAAGTAAAGTCGGTACTAACCAAAAGATGTTATTGACTATTCCAACTTC
TTCCACACCTTCTCCTTCCACTCATGCTGCCCCGGTAACACCCATCATTT
CTATACAGGAATTCAATGAGGGACACTTCCCAGTTAAAAACGAAGATGAC
GGAACGTTACAACTAAAAGTTCGGGATAACGAAAGTTATAGCGCTACTAA
CAATAACAATCTACTTCGTCCAGATGATAATGATTACAACAACGAAGCTC
TCAGTGATATTGACCGCTCCTTCGAAGATATCATTAACGGTCGAAAATTG
AAACTTAAAAAATCAAGAAGAAGATCTTCTCAAACTTCCAATAATAGTTT
CACTAGCAGGAGATCCTCGAGATCAAGAAGCATATCTCCCGATGAAAAAG
CTAAATCTATAAGTGCAAATAGGGAAAAGTTATTGGAGATGGCAGATCTT
TTACCATCCAGTGAAAATGACAATAATCGGGAGCGCTATGACAATGATAG
CAAAACTAGCTATAACACCATTAACAGTAGCAATTTTAATGAGGATAATA
ATAATAACAATTTGTTAACAAGTAAACCAAAAATTGAATCGGGCATTGTC
AATATTAAAAATGAACTAGATGACACTAGCAAAGATCTTGGCATACTTCT
AGACATAGATAGCTTGGGCCAATTTGAGCAGAAAGTTGGTTTCAAAAATG
ATGATAATCACGAAAATAACGATAATGGCACATTTTCTGTTAAGAAAAAT
GACAATCTAGAAAAACTAGACAGTGTAACAAATAATAGGAAAAATCCTGC
GAATTTTGCTTGTGATGTATGTGGTAAGAAGTTTACAAGACCTTATAACT
TAAAGTCACACTTAAGAACGCATACAAATGAAAGGCCATTTATTTGTTCT
ATTTGTGGTAAGGCGTTTGCACGTCAACATGATAGAAAGAGACACGAAGA
TTTGCATACAGGTAAGAAAAGGTATGTATGCGGTGGTAAGCTGAAGGACG
GTAAACCCTGGGGTTGTGGCAAAAAGTTTGCAAGAAGTGATGCTCTTGGT
AGGCATTTTAAAACTGAAAGTGGTAGAAGATGCATCACTCCCTTGTACGA
AGAAGCCAGACAGGAGAAATCGGGACAAGAGAGTTAA

### Example 2

### Generation of Modified CRZ1 Expression Cassettes

The native S. cerevisiae S288c CRZ1 gene was PCR-amplified from a genomic DNA preparation with upstream primer containing a 5′-SpeI site and a single nucleotide exchange to delete the CRZ1 internal SpeI site as previously described and downstream primer containing a 3′-NotI site. The amplification product along with plasmid pJT801 was digested with SpeI and NotI. Following incubation of the digested PCR product with the digested plasmid pJT801 in the presence of DNA ligase, a previously cloned GOI was replaced with the internal SpeI-deleted CRZ1, to be referred to as CRZ1.1. The new plasmid construct, pJT852 contains the DAL80 promoter::CRZ1.1::FBA1 terminator cassette.

The aforementioned modifications (i.e., serine to alanine substitutions) in the SRR of CRZ1 were introduced into the new plasmid by gBlock double-stranded DNA synthesis. The modified sequence was synthesized with upstream and downstream SpeI and PstI sites, respectively and used to replace the SpeI and PstI fragment of plasmid pJT852 to construct plasmid pJT860 containing the DAL80 promoter::CRZ1.1.2::FBA1 terminator expression cassette, to be referred to as CRZ1.2.

### Example 3

### Generation of Yeast Expressing Modified CRZ1

Plasmid pJT860 from Example 2 was used as a template for PCR amplification of the a CRZ1.1.2 expression cassette using appropriate flanking primers having homology to the AAP1 locus of S. cerevisiae. The amplified DNA fragment was used as donor DNA for CRISPR-mediated integration at the AAP1 locus in three parental strains: (i) FG is FERMAX™ Gold Label (Martrex Inc., Minnesota, USA; herein abbreviated, “FG”), (ii) FG-PKL is an engineered FG yeast having a heterologous phosphoketolase (PKL) pathway involving the expression of phosphoketolase (PKL), phosphotransacetylase (PTA) and acetylating acetyl dehydrogenase (AADH), as described in WO2015148272 (Miasnikov et al.), and (iii) FG-PKL-GA is the FG-PKL strain further engineered to expresses an exogenous glucoamylase (GA). The exogenous GA expressed by all yeast was the same variant of Trichoderma glucoamylase. Integration of the expression cassettes were confirmed by PCR.

### Example 4

### Effect of Modified CRZ1 Expression on Ethanol Production

Two clones of each strain expressing the integrated CRZ1.1.2 were screened for ethanol production, relative to control strains expressing only native CRZ1, by anaerobic growth conducted in Ankom flasks in corn liquifact growth medium. The ethanol production was analyzed at the end of a 55-hour fermentation. Note that each of the following pairs of data represent independently controlled experiments and should not be compared with each other.

TABLE 2
Ethanol production by variants

| | | Ethanol | Ethanol |
|---|---|---|---|
| Strain | Features | (g/L) | (% increase) |
| FG | FERMAX ® Gold | 144.13 | -0- |
| GJT324 | FG + CRZ1.1.2 | 144.67 | 0.37 |
| FG-PKL | PKL pathway | 145.27 | -0- |
| GJT204 | PKL pathway + CRZ1.1.2 | 146.75 | 1.02 |
| FG-PKL-GA | PKL pathway + GA | 147.75 | -0- |
| GJT276 | PKL pathway + GA + CRZ1.1.2 | 149.18 | 0.97 |

Increased ethanol production of up to over 1.0% was observed with modified CRZ1.

What is claimed is:

1. Modified yeast cells derived from parental yeast cells, the modified cells comprising a genetic alteration that causes the cells to produce an increased amount of active CRZ1 polypeptides compared to the parental cells, wherein the modified cells produce during fermentation an increased amount of alcohol compared to the amount of alcohol produced by the parental cells under identical fermentation conditions.

2. The modified cells of claim 1, wherein the active CRZ1 polypeptides exhibit reduced phosphorylation compared to native CRZ1 polypeptides under identical fermentation conditions.

3. The modified cells of claim 1 or 2, wherein the active CRZ1 polypeptides include a reduced number of amino acid residues capable of phosphorylation compared to the amino acid residues in native CRZ1 polypeptides.

4. The modified cells of claim 3, wherein the active CRZ1 polypeptides include a reduced number of serine residues capable of phosphorylation compared to the amino acid residues in native CRZ1 polypeptides.

5. The modified cells of any of claims 1-4, wherein the amount of increase in the expression of the modified CRZ1 mutant polypeptides is at least about 500%, at least 1,000%, at least 1,500%, or even at least 2,000%, compared to the level expression of native CRZ1 polypeptides in the parental cells grown under equivalent conditions.

6. The modified cells of any of claims 1-5, wherein the cells further comprising one or more genes of the phosphoketolase pathway.

7. The modified cells of claim 6, wherein the genes of the phosphoketolase pathway are selected from the group consisting of phosphoketolase, phosphotransacetylase and acetylating acetyl dehydrogenase.

8. The modified cells of any of claims 1-7, wherein the cells further comprise an exogenous gene encoding a carbohydrate processing enzyme.

9. The modified cells of any of claims 1-8, further comprising an alteration in the glycerol pathways and/or the acetyl-CoA pathway.

10. The modified cells of any of claims 1-9, further comprising an alternative pathway for making alcohol.

11. The modified cells of any of claims 1-10, wherein the cells are of a Saccharomyces spp.

12. The modified cells of any of claims 1-11, wherein the alcohol is ethanol.

13. A method for increasing the production of alcohol from yeast cells grown on a carbohydrate substrate, comprising: introducing into parental yeast cells a genetic alteration that increases the production of active CRZ1 polypeptides compared to the amount produced in the parental cells.

14. The method of claim 13, wherein the active CRZ1 polypeptides exhibit reduced phosphorylation compared to native CRZ1 polypeptides under identical fermentation conditions.

15. The method of claim 13 or 14, wherein the active CRZ1 polypeptides include a reduced number of amino acid residues capable of phosphorylation compared to the amino acid residues in native CRZ1 polypeptides.

16. The method of claim 15, wherein the active CRZ1 polypeptides include a reduced number of serine residues capable of phosphorylation compared to the amino acid residues in native CRZ1 polypeptides.

17. The method of any of claims 13-16, wherein the genetic alteration results in the variant yeast of any of claims 1-11, or variants, thereof.
